(19) United States
(12) Patent Application Publication
(10) Pub. No.: US 2006/0094000 A1
(43) Pub. Date: May 4, 2006

(54) Destructible surfactants and uses thereof

(76) Inventors: Claude R Mallet, Attleboro, MA (US); Reb J Russell, Manlius, NY (US); Kurt Yardley, San Diego, CA (US)

Correspondence Address: EDWARDS & ANGELL, LLP, P.O. BOX 55874, BOSTON, MA, 02205, US

(21) Appl. No.: 10/516,419

(22) PCT Filed: May 30, 2003
(86) PCT No.: PCT/US03/16819

§ 371 Date: Aug. 29, 2005

Related U.S. Application Data
(60) Provisional application No. 60/385,018, filed on May 31, 2002.

(51) Int. Cl. C12Q 1/00; G01N 33/00

(52) U.S. Cl. 435/4; 436/111

(57) ABSTRACT

Destructible surfactants and methods of using same are provided. The invention includes anionic surfactants having a dioxolane or dioxane functional group that enable degradation of the surfactant under acidic conditions. The invention also includes methods of using anionic surfactants in a variety of applications relating to samples containing small molecules.

### REFERENCE TO RELATED APPLICATIONS

This application claims priority to U.S. Provisional Patent Application No. 60/385,018, filed on May 31, 2002 (Attorney Docket No. WCZ-031-1). This application is related to U.S. Provisional Patent Application No. 60/134,113, filed on May 14, 1999 (Attorney Docket No. WCZ-004-1), and published PCT International application No. WO 00/70334, published Nov. 23, 2000 (Attorney Docket No. WAA-213 PCT; application No. PCT/US00/13028, filed on May 12, 2000). The entire contents of the aforementioned applications are hereby incorporated herein by reference.

### BACKGROUND OF THE INVENTION

The isolation and subsequent characterization of small molecules from cells, e.g., cell lysates, tissue cultures, e.g., culture supernatants, and/or biological fluids, e.g., blood plasma or urine, biological matrices, e.g., blood or bone, have been attempted with limited success using known methodology. The current methodology used in the analysis of small molecules from biological matrices, e.g., low-level drugs from complex mixtures, i.e., in toxicological screens for pharmaceutical drugs, is a lysis technique involving the use of organic solvents or known surfactants as described in Grosse, P. Y. et. al. (1997). High-performance liquid chromatographic assay for methyl-β-cyclodextrin in plasma and cell lysates. J. Chrom. B. 694:219-226.

In methods that utilize organic solvents, the organic solvent must be removed, or diluted from a sample, prior to a typical solid phase extraction workup step. Removal of the solvent is typically accomplished through subjection of the sample to an evaporation process that tends to be both manual in nature and lengthy in time. However, dilution of the sample tends to prohibit complete recovery of the small molecule from the sample, increase the sample load on the system, and increase the overall length of time of the analysis.

In those methods that use surfactants, e.g., sodium dodecylsulfate (SDS) or Triton X-100, the surfactant must be removed prior to mass spectrometric analysis to prevent ion suppression of the analyte of interest through extensive cleaning, or purification. This cleaning procedure causes an increase in the overall length of time required for the analysis.

Moreover, regardless of the methodology, existing processes require additional time-consuming and tedious sample preparation steps that result in time and sample loss, making accurate analysis of low-level compounds difficult. Furthermore, as a result of the need for additional steps, existing techniques for analysis of small molecules are not optimal for automation.

### SUMMARY OF THE INVENTION

The present invention features destructible surfactants and methods for analyzing (e.g., solubilizing, analyzing, separating, isolating, purifying, detecting and/or characterizing) small molecules contained in samples (e.g., from cells, e.g. cell lysates, tissue cultures, e.g., culture supernatants, and/or biological fluids, i.e., biological matrices, e.g., blood plasma or urine) using these surfactants. In one aspect, the anionic surfactants of the present invention may be selectively broken up at relatively low pH. The resulting breakdown products of the surfactants may be removed from the sample with relative ease.

The invention has applicability in a variety of techniques which benefit from the initial presence and ultimate removal of a surfactant. Moreover, the surfactants of the present invention eliminate the need for organic solvents and surfactants in the methodology of sample analysis. The elimination of the time-consuming and tedious sample preparation steps that result in both time and sample loss, allow for accurate analysis of low-level compounds. As a result, the techniques for analysis of small molecules of the present invention are well suited for automation.

In particular, the methods of the invention provide for and/or facilitate analysis of small molecules in samples taken from biological matrices, including Adsorption, Distribution, Metabolism, Excretion (ADME), metabolomics, bioanalytical analysis, and pharmacokinetics. Thus, the invention is useful, e.g., in clinical studies for therapeutic molecules, toxicology studies and animal modeling.

Accordingly, in one aspect, the invention provides methods for analysis of a small molecule, which includes contacting the sample containing at least one small molecule with a surfactant represented by the formula (Formula I): 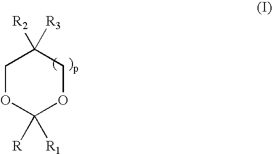
in which
p is 0, 1 or 2;
R is alkyl;
R₁ and R₂ are each, independently, hydrogen or methyl; and
R₃ is selected from —OSO₃⁻, —R₄OSO₃⁻, —R₄OR₅SO₃⁻, and —OR₅SO₃⁻,
wherein R₄ and R₅ are each, independently, lower alkyl; to thereby analyze the small molecule.

Another aspect of the invention provides a method for performing cell lysis comprising contacting a cell containing at least one small molecule with a surfactant having the structure of Formula I, to thereby lyse the cell.

In yet another aspect, the invention provides a kit for performing cell lysis on a sample containing at least one small molecule to isolate the small molecule comprising a surfactant having the structure of Formula L and instructions for use.

In another aspect, the invention provides a method for electrophoretically isolating a small molecule from a sample. The method includes contacting a sample containing at least one small molecule with a surfactant having the structure of Formula I to form a sample/surfactant complex, and performing electrophoresis on the sample/surfactant complex, to thereby electrophoretically isolate the small molecule.

In an additional aspect, the invention provides a kit for performing electrophoresis on a sample containing at least one small molecule, which includes a surfactant having the structure of Formula I, and instructions for use.

In another aspect, the invention provides a method of solubilizing a small molecule comprising contacting a sample containing at least one small molecule with a surfactant having the structure of Formula I.

In yet another aspect, the invention provides a method of regenerating a liquid chromatography column having a sorbent to which is bound at least one small molecule, comprising contacting the sorbent with a surfactant having the structure of Formula I, such that small molecule bound to the sorbent is removed, thereby regenerating the column.

In an additional aspect, the invention provides a method for analyzing a small molecule contained in a cell comprising contacting the cell with a surfactant represented by the formula (Formula I), to lyse the cell, and analyzing the small molecule.

### Brief DESCRIPTION OF THE DRAWINGS

FIG. 1 depicts the electrospray mass spectra of propranolol under the various treatment conditions described in Example 2. FIG. 1 also shows a comparison of the MS analysis of the cell lysates using ALS as compared with SDS.

FIG. 2 depicts the on-line cartridge/column configuration, connected to a 10-port switching valve and peripherals (2700, 515 pump, 2690 and Quatttro Ultima).

FIG. 3 depicts the HPLC and gradient wash conditions used for analysis of the cell pellet spiked with propranolol and lysed with organic solvent.

FIG. 4 depicts the results for the analysis of the cell pellet spiked with propranolol and lysed with organic solvent.

FIG. 5 shows the general ion suppression in the detection using mass spectrometry when the sample contains no surfactant, 0.5% SDS (in water), or 0.5% Triton X100.

FIG. 6 depicts the on-line cartridge/column configuration with two cartridges (one filled with Oasis MCX and the other filled with Oasis HLB) connected in series with 4 switching valves and peripherals.

FIG. 7 depicts the HPLC and gradient wash conditions used for analysis of the cell pellet spiked with propranolol and lysed with surfactant.

FIG. 8 depicts the results for the analysis of the cell pellet spiked with propranolol, lysed with Triton X-100, and washed with 100% methanol.

FIG. 9 depicts the results for the analysis of the cell pellet spiked with propranolol, lysed with SIDS, and washed with 100% methanol.

FIG. 10 depicts the results for the analysis of the cell pellet spiked with propranolol, lysed with SDS, and washed with 50/50 acetonitrile and acetone.

### DETAILED DESCRIPTION OF THE INVENTION

Overview of the Invention

The invention provides anionic surfactants, including the use of anionic surfactants in the analysis (e.g., solubilizing, analyzing, separating, isolating, purifying, detecting and/or characterizing) of small molecules from cells, e.g., cell lysates, tissue cultures, e.g., culture supernatants, and/or biological fluids, i.e., biological matrices, e.g., blood plasma or urine. In particular, the invention includes anionic surfactants with binding and electrophoretic properties similar to sodium dodecylsulfate (SDS). Unlike SDS, however, the surfactants of the present invention include a dioxolane or dioxane functional group that enables degradation of the surfactant under an acidic environment. The resulting degradant products can be removed from the sample more readily than the original surfactant. In addition, mass spectrometric sensitivity of the small molecules is significantly and surprisingly greater in the presence of the surfactants of the invention than in the presence of SDS at similar concentrations, even in the presence of these degradant products.

Examples of applications which will benefit from this invention include, without limitation, electrophoresis, ion-pair liquid chromatography, liquid chromatography, mass spectrometric detection, e.g., MALDI-MS or electrospray, liquid-liquid extraction, and other techniques which benefit from the initial presence and ultimate removal of a surfactant.

Definitions

Before further description of the invention, certain terms employed in the specification, examples and appended claims are, for convenience, collected here.

The language “sample/surfactant complex” is intended to include a complex formed by a surfactant of the present invention and a component of the sample.

The term “sample” refers to any solution of a molecule or mixture of molecules that comprises at least one small molecule that is subjected to analysis. Particular examples include, but are not limited to, biological samples. The sample may further include macromolecules, e.g., substances, such as biopolymers, e.g., proteins, e.g., proteolytic proteins or lipophilic proteins, such as receptors and other membrane-bound proteins, and peptides.

The language “biological sample” refers to any solution or extract containing a molecule or mixture of molecules that comprises at least one biomolecule that is subjected to analysis that originated from a biological source. Biological samples are intended to include crude or purified, e.g., isolated or commercially obtained, samples. Particular examples include, but are not limited to, inclusion bodies, biological fluids, biological tissues, biological matrices, embedded tissue samples, cells (e.g., one or more types of cells), and cell culture supernatants.

The language “biological matrices” is intended to include anything that a cell contains or makes, e.g., bone, inclusion bodies, blood components, cells, e.g., cell lysates, etc.

The language “biological fluid” as used herein is intended to include fluids that are obtained from a biological source. Exemplary biological fluids include, but are not limited to, blood, blood plasma, urine, spinal fluid, mucosal tissue secretions, tears, interstitial fluid, synovial fluid, semen, and breast milk.

The language “small molecules” is intended to include all molecules that are less than about 1000 atomic mass units (amu). In certain embodiments of the invention, the small molecule is not a peptide. In other embodiments, the small molecule is an organic, non-proteinaceous molecule. In particular embodiments, the small molecule is a pharmaceutical drug, e.g., a low-level pharmaceutical drug, a pro-drug, a metabolite of a drug, or a product of a reaction associated with a natural biological process, e.g., enzymatic function or organ function in response to a stimulus.

The language “natural biological process” is intended to include a process that occurs naturally in the human body, which may or may not be functioning as it would be in a healthy person. In certain embodiments of the invention, the analysis of a product of a reaction associated with a natural biological process is used to determine whether the natural biological process is functioning properly. Moreover, the language natural biological process is not making a reference to the quality of the process that is occurring, but merely that the process occurs naturally in the human body.

The term “lipophilic protein” refers to proteins or peptides that are relatively hydrophobic. Particular examples include, without limitation, protein from myelin or central nervous system tissue and membrane-bound proteins such as receptors.

The term “receptor” is recognized in the art and refers generally to membrane-bound molecules, preferably proteins, which bind a ligand and transmit a signal into the cell. Such receptors usually have an extracellular domain, a transmembrane domain, and an intracellular domain.

The term “inclusion body” is recognized in the art and refers to an intracellular structure, preferably one containing an expressed protein.

The language “solution for degrading the surfactant” refers to any relatively low pH solution. Preferably, the pH of the solution is between about 0 and about 5, more preferably between about 1 and about 3. In general, the lower the pH of the solution for degrading the surfactant, the less time required to degrade the surfactant. In addition, the compound used to make the solution for degrading the surfactant is not particularly limited: any compound that provides a relatively low pH solution suitable for degrading the surfactants of the present invention without damaging the sample is sufficient. Thus, for example, hydrochloric acid, acetic acid, formic acid, or trifluoroacetic acid (TFA) may be used as the solution for degrading the surfactant. In particular embodiments, TFA may be used to degrade the surfactant. In other particular embodiments, acetic or formic acid may be used as the solution for degrading the surfactant.

The terms “analysis” or “analyzing” are used interchangeably and refer to any of the various methods of separating, detecting, isolating, purifying, solubilizing, detecting and/or characterizing small molecules (e.g., pharmaceutical drugs). Examples include, but are not limited to, solid phase extraction, solid phase micro extraction, electrophoresis, mass spectrometry, e.g., MALDI-MS or ESL liquid chromatography, e.g., high performance, e.g., reverse phase, normal phase, or size exclusion, ion-pair liquid chromatography, liquid-liquid extraction, e.g., accelerated fluid extraction, supercritical fluid extraction, microwave-assisted extraction, membrane extraction, soxhlet extraction, precipitation, clarification, electrochemical detection, staining, elemental analysis, Edmund degradation, nuclear magnetic resonance, infrared analysis, flow injection analysis, capillary electrochromatography, ultraviolet detection, and combinations thereof.

The term “electrophoresis” refers to any of the various methods of analyzing small molecules by their rate of movement in an electric field, i.e. based on the charge to mass ratio of the molecules. Examples include, but are not limited to, free zone electrophoresis and capillary electrophoresis.

The term “clarification” refers to any process by which insoluble particulate matter is separated from the liquid phase.

The term “mass spectrometric detection” refers to any of the various methods of mass spectroscopy. Examples include, but are not limited to, electrospray ionization (“ESI”), surface desorption ionization techniques, and atmospheric pressure chemical ionization (APCI).

The language “surface desorption ionization” is intended to include mass spectrometry, such as matrix assisted laser desorption ionization (MALDI-MS), desorption ionization on silicon (DIOS), thermal desorption mass spectrometry, or surface enhanced laser desorption ionization (SELDI) where desorption ionization is accomplished on a surface, with or without a matrix assistance.

The language “hydrocarbon” includes substituted or unsubstituted alkyl, alkenyl, alkynyl, or aryl moieties.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₂₀ for straight chain, C₃-C₂₀ for branched chain), and more preferably 20 or fewer. Likewise, preferred cycloalkyls have from 3-8 carbon atoms in their ring structure, and more preferably have 5 or 6 carbons in the ring structure.

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)). The term “alkyl” also includes the side chains of natural and unnatural amino acids.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₁-C₂₀ for straight chain, C₃-C₂₀ for branched chain). Likewise, cycloalkenyl groups may have from 3-8 carbon atoms in their ring structure, and more preferably have 5 or 6 carbons in the ring structure.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₁-C₂₀ for straight chain, C₃-C₂₀ for branched chain).

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to ten carbon atoms. “Lower alkenyl” and “lower alkynyl” have chain lengths of, for example, 2 to 6 carbon atoms, more preferably 3 or 4 carbon atoms.

The term “acyl” includes compounds and moieties that contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide” or “aminocarboxy” includes compounds or moieties that contain a nitrogen atom which is bound to the carbon of a carbonyl or a thiocarbonyl group. The term includes “alkaminocarboxy” groups which include alkyl, alkenyl, or alkynyl groups bound to an amino group bound to a carboxy group. It includes arylaminocarboxy groups which include aryl or heteroaryl moieties bound to an amino group which is bound to the carbon of a carbonyl or thiocarbonyl group. The terms “alkylaminocarboxy,” “alkenylaminocarboxy,” “alkynylaminocarboxy,” and “arylaminocarboxy” include moieties wherein alkyl, alkenyl, alkynyl and aryl moieties, respectively, are bound to a nitrogen atom which is in turn bound to the carbon of a carbonyl group.

The term “carbonyl” or “carboxy” includes compounds and moieties that contain a carbon connected with a double bond to an oxygen atom. Examples of moieties that contain a carbonyl include aldehydes, ketones, carboxylic acids, amides, esters, anhydrides, etc.

The term “thiocarbonyl” or “thiocarboxy” includes compounds and moieties that contain a carbon connected with a double bond to a sulfur atom. The term “ester” includes compounds and moieties that contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “ether” includes compounds or moieties that contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “thioether” includes compounds and moieties that contain a sulfur atom bonded to two different carbon or hetero atoms. Examples of thioethers include, but are not limited to althioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated,” e.g., perfluorinated, generally refers to a moiety, e.g., perfluorocarbons, wherein all hydrogens are replaced by halogen atoms, e.g., fluorine.

The terms “polycyclyl” or “polycyclic radical” refer to two or more cyclic rings (e.g., cycloalkyls, cycloalkenyls, cycloalkynyls, aryls and/or heterocyclyls) in which two or more carbons are common to two adjoining rings, e.g. the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

Compounds of the Invention

The destructible surfactants of the invention may be prepared as shown in Scheme 1 set forth in Example 1 below. These surfactants have functionality similar to SDS but, unlike SDS, may be hydrolyzed in aqueous acid solution under mild condition to give two nonsurfactant products: an ionic, water-soluble compound and a neutral, water-insoluble compound.

In one embodiment, the anionic surfactants of the invention have the structure of the general formula (Formula I): 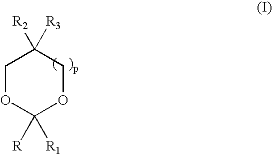
in which
p is 0, 1 or 2;
R is alkyl;
R₁ and R₂ are each, independently, hydrogen or methyl; and
R₃ is selected from —OSO₃⁻; —R₄OSO₃⁻, —R₄OR₅SO₃⁻, and —OR₅SO₃⁻,
wherein R₄ and R₅ are each, independently, lower alkyl.

In certain embodiments, the surfactants have the structure of Formula I, with the provisos that when p is 0 and R₁ is methyl, R₃ is not —CH₂O(CH₂)₄SO₃⁻ or, when p is 1 and R₁ is hydrogen and R₂ is methyl, R₃ is not —CH₂OSO₃.

In particular embodiments, p is 0 or 1. In other particular embodiments, R is an alkyl having from six to twenty carbon atoms, more specifically from eight to eighteen carbon atoms, and most preferably from ten to sixteen carbon atoms. In certain embodiments, R₃ is —R₄OSO₃⁻, —R₄OR₅SO₃⁻, or —OR₅SO₃⁻, and most preferably R₃ is —CH₂O(CH₂)₃SO3₃⁻ or —CH₂O(CH₂)₄SO3₃⁻. In certain embodiments, R₄ and R₅ are each, independently, an alkyl group having from one to eight carbons, more specifically from two to six carbon atoms, and more specifically, three or four carbon atoms.

In another embodiment, the anionic surfactants of the invention have the structure of general formula (Formula II):

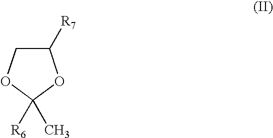

in which

R₆ is alkyl;

R₇ is selected from —OSO₃⁻, —R₄OSO₃⁻, —R₄OSO₃⁻, and —OR₅SO₃⁻,

wherein R₄ and R₅ are each, independently, lower alkyl.

In certain embodiments, the surfactants of the present invention have the structure of Formula II, with the proviso that when R₆ is —C₉H₁₉, —C₁₁H₂₃, or —C₁₃H₂₇, R₇ is not —CH₂O(CH₂)₄SO₃⁻.

In particular embodiments, the surfactant of the invention has the following chemical structure:

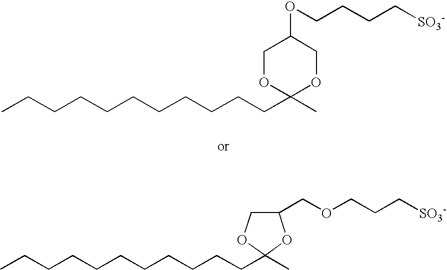

As indicated in more detail in the Examples, the methods of synthesis of the present invention produce isomers. Although the methods of using surfactants of the invention do not require separation of these isomers, such separation may be accomplished, if desired, by methods known in the art. For example, preparative high performance liquid chromatography methods may be used for isomer purification.

Methods of the Invention

The surfactants of the present invention may be used in applications that benefit from the initial presence and ultimate removal of a surfactant. In particular, the present invention is useful for the solubilization, analysis, separation, isolation, purification, detection and/or characterization of small molecules from biological samples, such as biological fluids, biological tissues, biological matrices, embedded tissue samples, and cell culture supernatants.

In one embodiment, the invention provides methods for analysis of a small molecule, which includes contacting the sample containing at least one small molecule with a surfactant of the present invention, to thereby analyze the small molecule. In certain embodiments, the sample may be heated either before or after contacting the sample with a surfactant of the invention. In certain embodiments, the step of analyzing the sample includes electrophoresis. In particular embodiments, the electrophoresis is free zone electrophoresis or capillary electrophoresis.

Analysis of the sample may include, without limitation, solid phase extraction, solid phase micro extraction, electrophoresis, mass spectrometry, e.g., MALDI-MS or ESI, liquid chromatography, e.g., high performance, e.g., reverse phase, normal phase, or size exclusion, ion-pair liquid chromatography, liquid-liquid extraction, e.g., accelerated fluid extraction, supercritical fluid extraction, microwave-assisted extraction, membrane extraction, soxhlet extraction, precipitation, clarification, electrochemical detection, staining, elemental analysis, Edmund degradation, nuclear magnetic resonance, infrared analysis, flow injection analysis, capillary electrochromatography, ultraviolet detection, and combinations thereof.

Another embodiment of the invention provides a method for performing cell lysis comprising contacting a cell containing at least one small molecule with a surfactant of the present invention, to thereby lyse the cell. In certain embodiments, analysis, e.g., mass spectroscopy or electrophoresis, is performed on the small molecule after cell lysis. In certain embodiments, the surfactant is degraded after electrophoresis. Degradation of the surfactant can be performed by contacting the surfactant with an acidic solution. In specific embodiments, the small molecule is purified, e.g., by solid phase extraction or HPLC after degradation of the surfactant.

In another embodiment, the invention provides a kit for performing cell lysis on a sample containing at least one small molecule to isolate the small molecule comprising a surfactant of the present invention, and instructions for use. In certain embodiments, the kit can additionally include a solution for degrading the surfactant or a solid phase extraction device.

An additional embodiment of this invention uses destructible surfactants of the invention to complex with mixtures, e.g., biological samples, e.g., cell cultures, containing at least one small molecule for electrophoresis. After the electrophoretic separation, the separated components, e.g., small molecules and proteins, are released from the surfactants of the present invention by treating with acid solution. The isolated small molecules may be further purified by conventional separation methods such as liquid-liquid extraction, solid-phase extraction or liquid chromatography. This ability to release the small molecules from surfactants easily after electrophoresis may be used in various applications, with significant benefits to separation science.

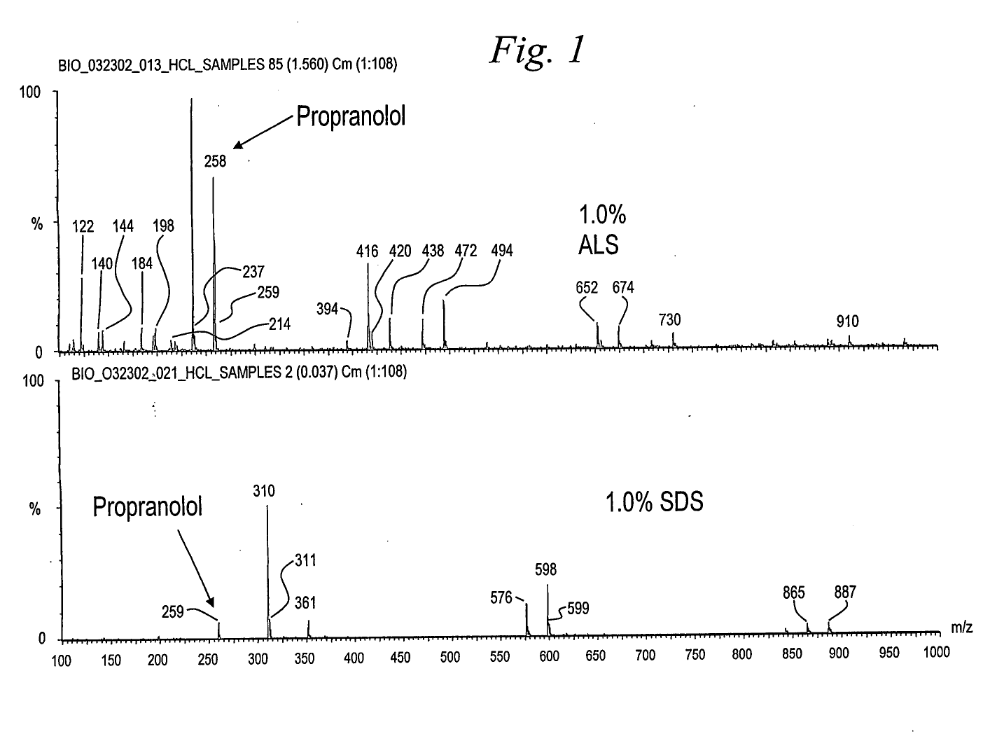

In accordance with the invention, the sensitivity of mass spectrometric detection of small molecules in the presence of degraded ALS is much greater than in the presence of SDS. The anionic surfactants of the present invention provide surprising advantages over SDS when analyzing small molecules. For example, in FIG. 1B, which is the mass spectrum of propranolol treated with SDS, no signals due to propranolol are observed. In contrast, as seen in FIG. 1A, the mass spectrum of propranolol treated with a surfactant of the present invention, after degradation, exhibits a strong propranolol signal. Without wishing to be bound by any particular theory, this result is believed to be due to at least two effects: 1) few, if any, micelles are present with the degraded surfactant of the present invention; and 2) fewer adducts of sample and the degraded surfactant of the invention are formed. These effects allow better sensitivity in mass spectrometry than is possible when SDS is used.

In still another embodiment, the invention provides a kit for performing electrophoresis on a sample containing at least one small molecule, which includes a surfactant of the present invention, and instructions for use. In a particular embodiment, the kit includes a component for degrading the surfactant. In another particular embodiment, the kit includes a molecular weight standard. In still another particular embodiment, the kit includes a staining reagent.

In another aspect, the invention provides a method of solubilizing a small molecule comprising contacting a sample containing at least one small molecule with a surfactant of the present invention.

In yet another embodiment, the invention provides a method of regenerating a liquid chromatography column having a sorbent to which is bound at least one small molecule comprising contacting the sorbent with a surfactant of the present invention, such that small molecule bound to the sorbent is removed, thereby regenerating the column. In a particular embodiment, a surfactant having the structure of Formula I is contacted with the sorbent of an HPLC column or solid phase extraction device, such that small molecules bound to the column are removed.

In an additional embodiment, the invention provides a method for analyzing a small molecule contained in a cell comprising contacting the cell with a surfactant of the present invention to lyse the cell, and analyzing the small molecule. In certain embodiments of the invention, the step of analyzing comprises mass spectrometry or electrophoresis.

### EXEMPLIFICATION

The invention is further illustrated by the following examples that should not be construed as limiting.

### Example 1

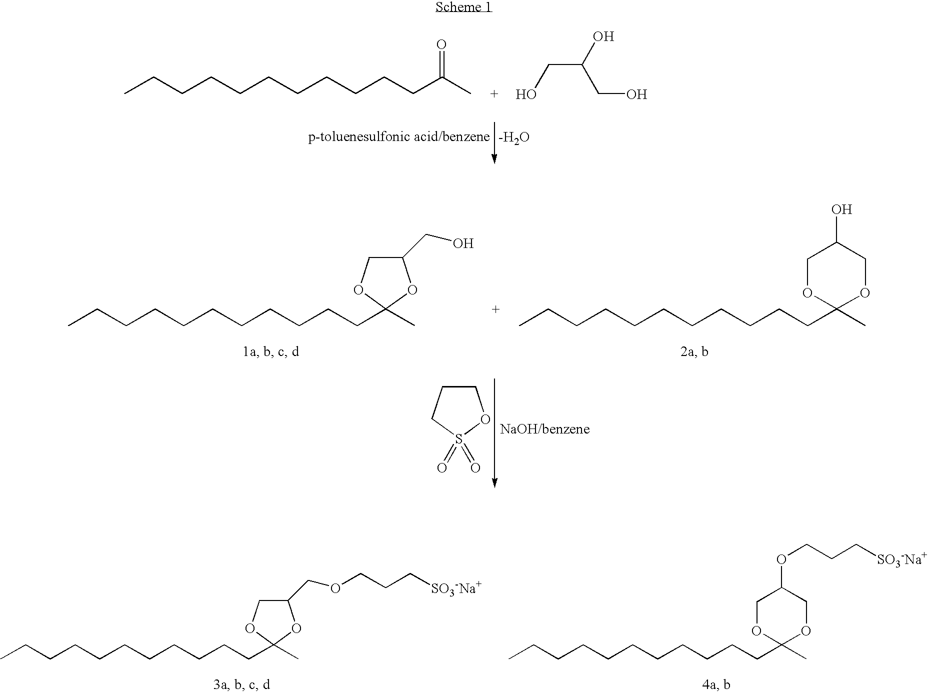

This example describes the preparation of certain anionic surfactants of the present invention. Various modifications to the following procedures will be routine to one of ordinary skill in the art, in light of the teachings herein. For example, in the following procedures, toluene may be substituted for benzene. In addition, any solvent that provides a sufficient yield may be used in the recrystallization step.

1. Synthesis of 4 hydroxymethyl-2-methyl-2-undecyl-1,3-dioxolane (1, 2)

Firstly, 100 g (0.5 mol) of 2-tridecanone (Aldrich P/N 17,283-9), 56 g (0.6 mol) of glycerol (Aldrich P/N 32,00-5), 200 mL of benzene, and 1.8 grams of p-toluenesulfonic acid (Aldrich P/N 40,2885) were placed in a 500 mL round bottom flask fitted with a Dean Stark apparatus. The mixture was heated to reflux with stirring until no further separation of water appeared. The reaction mixture was cooled to room temperature and washed successively with a 100 mL portion of 5% sodium carbonate solution and three 100 mL portions of water. The organic layer was dried over sodium sulfate, filtered and the benzene was removed with a rotary evaporator. The residual oil was fractionated by distillation under reduced pressure to give the desired product (b.p. 140° C./0.3 mm Hg). The identity of the product was confirmed by H NMR in CDCl₃.

2. Synthesis of ALS:

50 g (0.18 mol) of 4-hydroxymethyl-2 methyl-2 undecyl-1,3-dioxolane, 8 g (0.2 mol) of powdered sodium hydroxide and 200 mL of benzene were placed in a 4 neck 500 mL flask fitted with a condenser, mechanical stirrer and a thermometer. The suspension was stirred at a constant 50° C. while 25 g (0.2 mol) of 1,3-propanesultone (Aldrich P/N P5,070-6) was slowly added over 30 minutes. The suspension was then stirred at 70-75° C. for at least 6 hours. Upon completion, the reaction mixture was poured into 500 mL of boiling ethanol. The volume of the resulting mixture was then reduced in vacuo with a rotary evaporator, producing a solid residue that was subsequently dissolved in boiling ethanol and hot filtered.

The solid residue was additionally extracted with boiling ethanol, which was combined with the mother liquor. The solvent was removed in a rotary evaporator, and the resulting residue was then recrystallized from ethanol to yield the product. Identity of the product was confirmed by ¹H NMR in D₂O.

### Example 2

### Cell Lysis and Mass Spectrometric Detection of Propranolol Treated with ALS Compared to Organic Solvent, SDS, or Triton X100 Lysis

Jurkat cells (10⁶ cells) were grown in RPMI 1640 culture medium. The cells were then centrifuged at 1500 g for 5 minutes and subsequently washed twice with cold phosphate buffered saline (PBS). The cells were then centrifuged at 1500 g for an additional 5 minutes, and the supernatant was removed. The resulting cell pellets were spiked at various levels of propranolol (structure shown below).

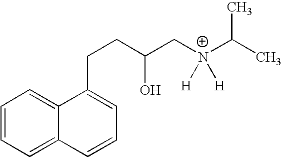

The cells of the pellet were lysed with (1) 1 mL 50/50 MeOH/ACN, (2) 1 mL 1%. SDS (aqueous), (3) 1 mL 1% Triton X100 (aqueous), or (4) 1 mL 1% ALS (aqueous), followed by dilution with 4 mL H₂O. The samples (400 μL) were injected onto a hydrophilic-lipophilic balanced copolymer solid phase extraction device, i.e., an on-line Oasis® HLB and MCX Extraction Column, 2.1 mm LD.×20 mm 25 μm, fitted with an MS/MS hyphenated system.

(1) 1 mL 50/50 MeOH/ACN

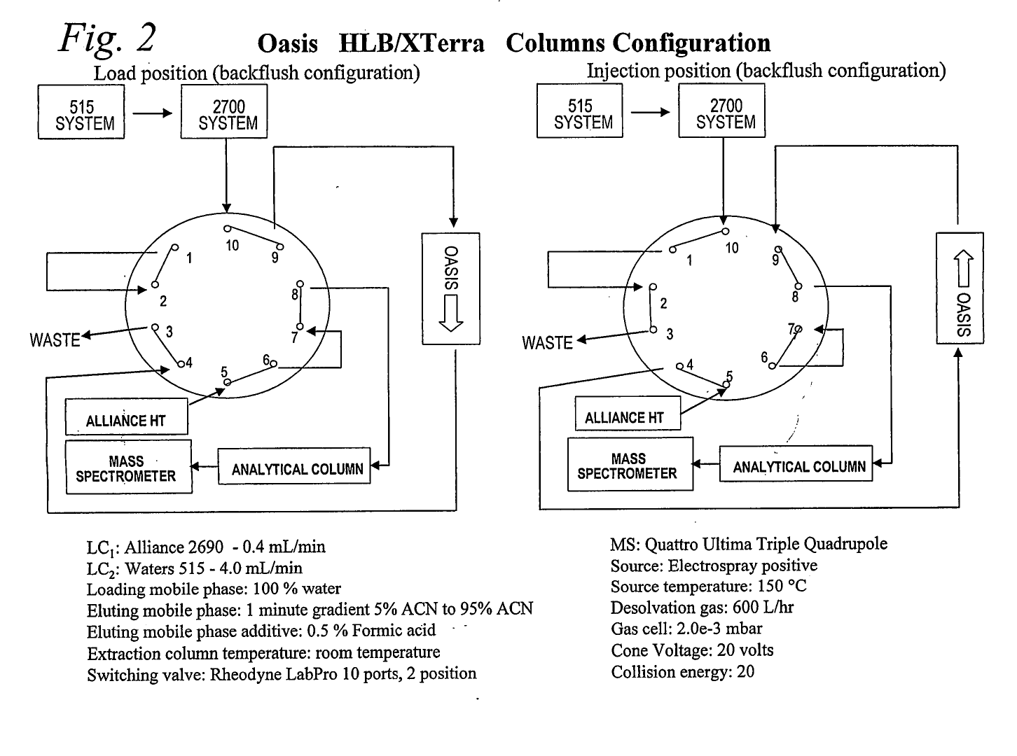

The first technique studied involved lysing the cell pellet with an organic solvent mixture. In this study, cells were lysed with 50/50 MeOH/ACN and were analyzed using a single solid phase extraction (SPE) cartridge (2.1×20 mm) filled with Oasis HLB. The on-line cartridge was connected to a 10-port switching valve and peripherals (2700, 515 pump, 2690 and Quatttro Ultima) as shown in FIG. 2. The analysis was carried out as follow:

Load step: 1.5 mL of sample was injected at high flow rate (4 mL/min) into an Oasis HLB cartridge for 30 seconds using a 100% aqueous mobile phase. The 515 pump was used as a stand-alone unit to deliver a constant flow rate through the cartridge, with the switching valve in a loading position with the effluent diverted to a waste line. Meanwhile, the 2690 pump delivered a constant 0.4 mL/min flow rate through the second circuit of the switching valve to the electrospray source of the mass spectrometer.

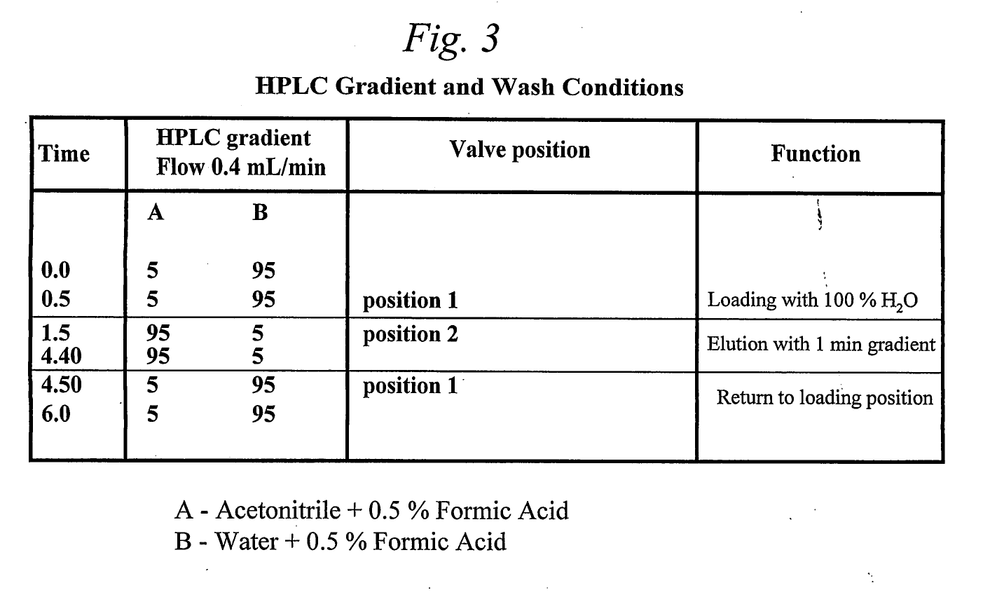

Elution step: After 30 seconds, the switching valve was pulsed to its second position. The effluent from the 2690 was redirected through the Oasis cartridge and the gradient was activated at the same time. The effluent of the 515 pump was discarded to a waste line until the analysis was completed. The gradient was ramped over a period of 1 minute from 5% organic (Acetonitrile with 0.5% formic acid) to 95% organic. The high organic level was kept for an additional 2.9 minutes before returning to its original condition at 4.5 minutes, as shown in FIG. 3.

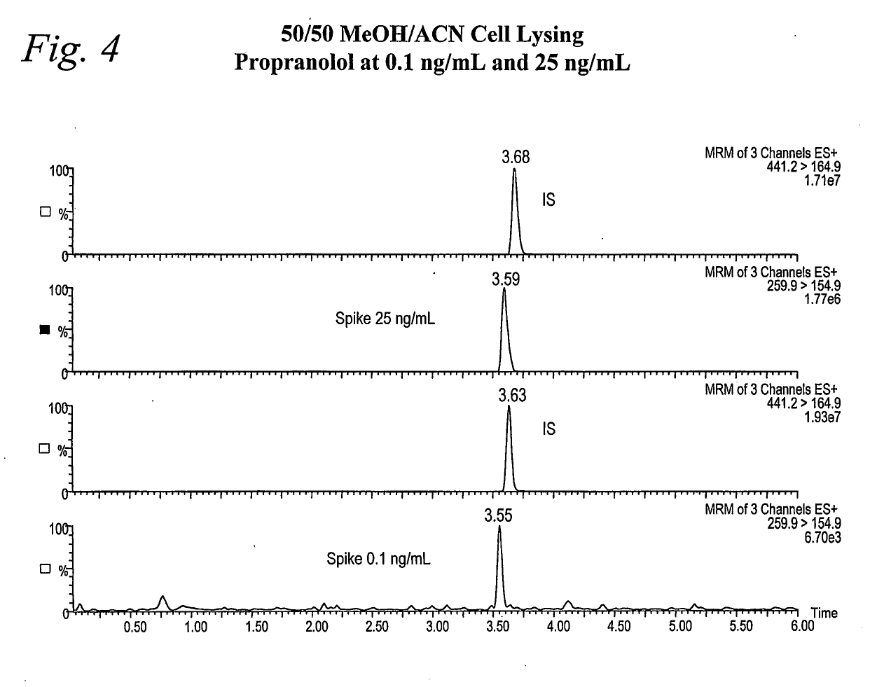

The results for the analysis of the sample lysed with organic solvents, shown in FIG. 4, demonstrate sensitivity as low as 0.1 ng/mL. The baseline at this concentration is stable and indicates that the extraction protocol gives a high level of clean-up capability. However, prior to the analysis the cell lysate requires a dilution step with water to ensure that the propranolol will bind to the sorbent. In the absence of this dilution step, breakthrough of the drug (no retention on the sorbent) would likely occur. Moreover, the dilution step significantly decreases the sensitivity of the analysis.

(2) 1% SDS (Aqueous), (3) 1% Triton X100 (Aqueous), and (4) 1% ALS

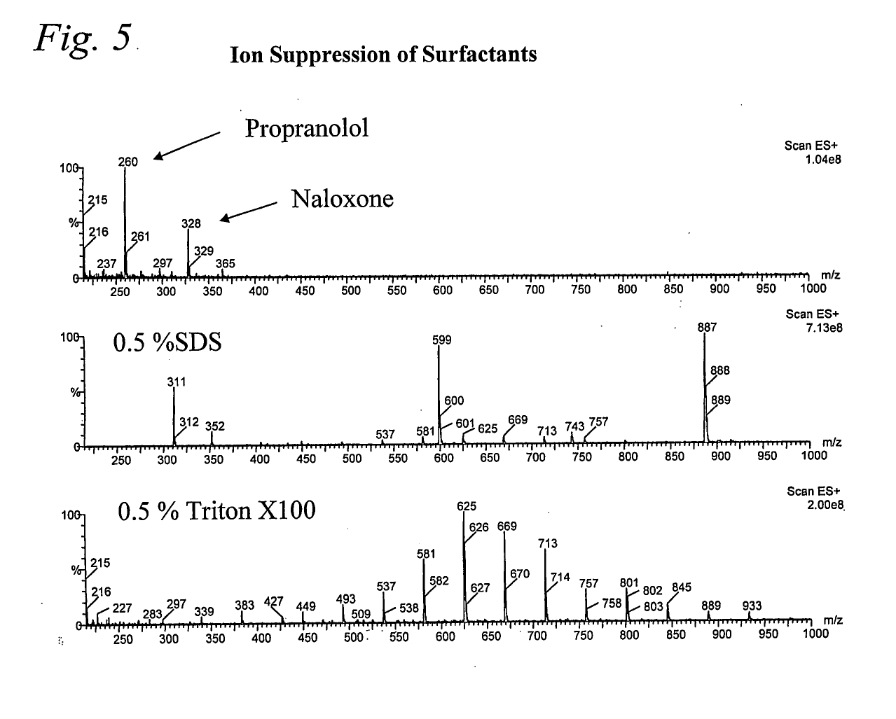

The second lysing technique studied involved the use of a surfactant rather than an organic solvent. Surfactants are known to be ion suppressant, especially in ESI mass spectrometry (either in positive or negative), as can be seen in FIG. 5. Because ion suppression is compound dependent, trace amount of surfactants can cause a total loss of signal at low levels and reduced signals for higher concentrations. Therefore, a lysing technique using a known surfactant will typically require a strong clean-up protocol. Moreover, ion exchangers such as SPE sorbent have been shown to give better results and recoveries than a reversed phase sorbent.

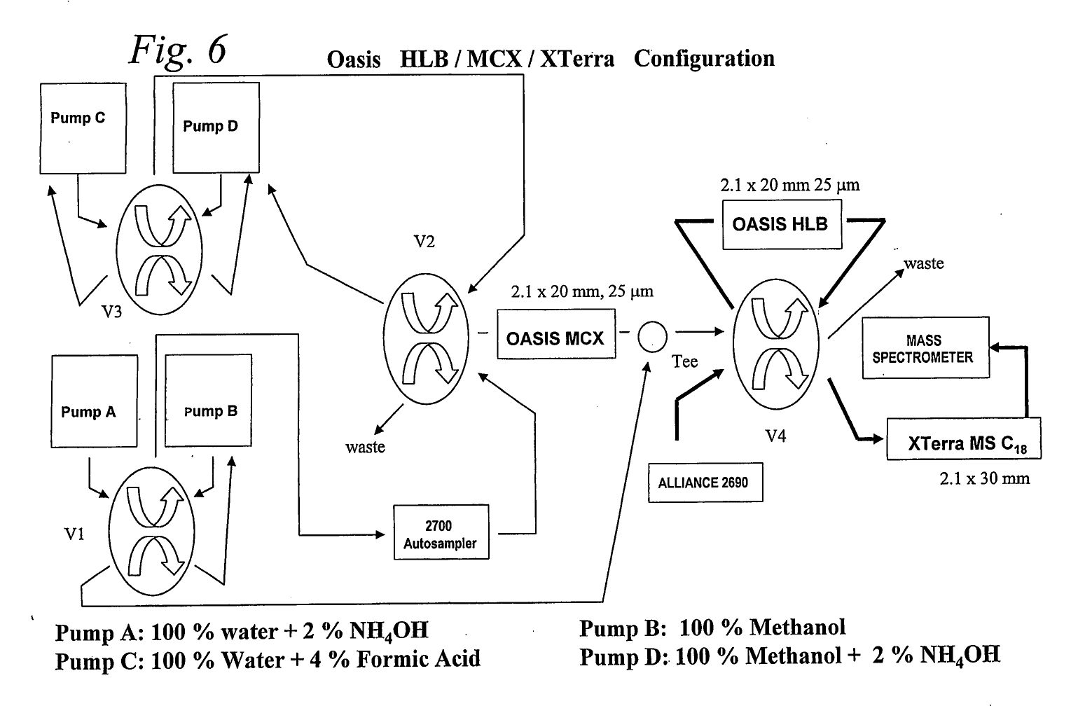

In this study, two typical surfactants were evaluated: an ionic surfactant, SDS, and a neutral surfactant, Triton X100. In this experiment, two cartridges (one filled with Oasis MCX and the other filled with Oasis HLB) were connected in series with 4 switching valves and peripherals, as shown in FIG. 6. The analysis was carried out as follow:

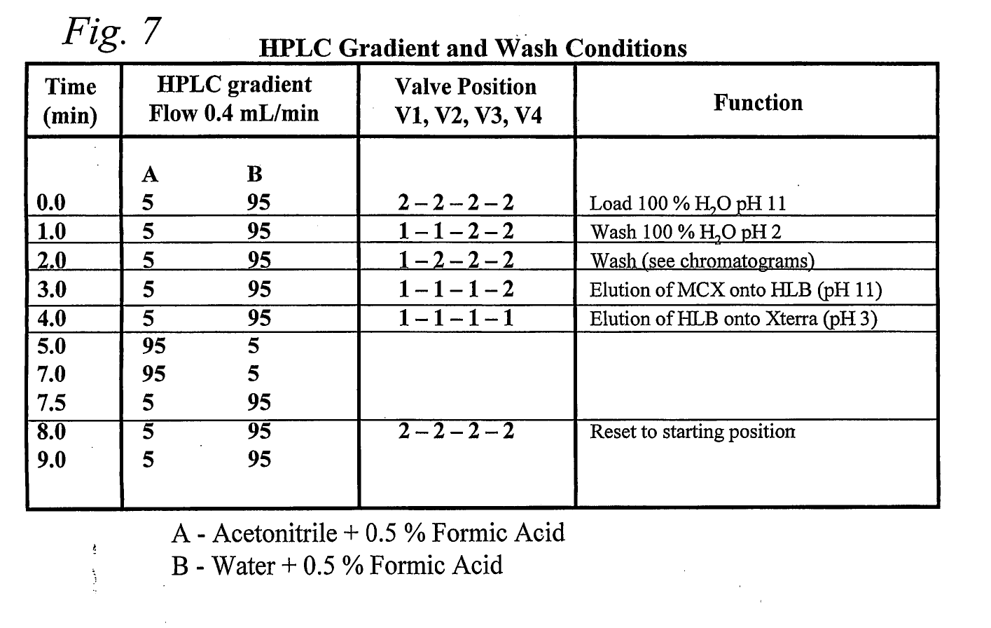

Loading step: 1.5 mL of sample was loaded on the MCX cartridge at 4 mL/min using a 100% aqueous mobile phase with 2% ammonium hydroxide (pump A) and the line was directed to a waste container to ensure that the propranolol will be retained as a neutral species on the reversed phase of the MCX sorbent. After one minute of loading, the LC program was switched to the appropriate valve for the next step, depicted in FIG. 7.

Washing step #1: The effluent of pump C was directed toward the Oasis MCX cartridge for the first wash according to the valve switching program, i.e., for one minute, to ionize any propranolol trapped on the reversed phase of MCX and “lock” it onto the ion exchanger. The mobile phase was 100% water plus 4% formic acid and set a 4 ml/min.

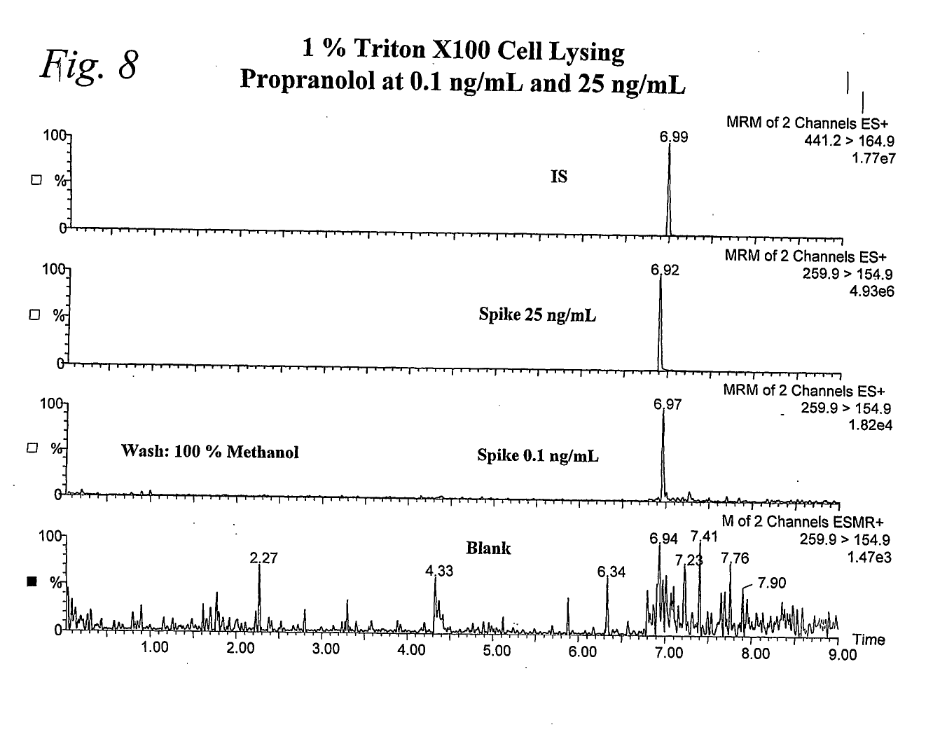

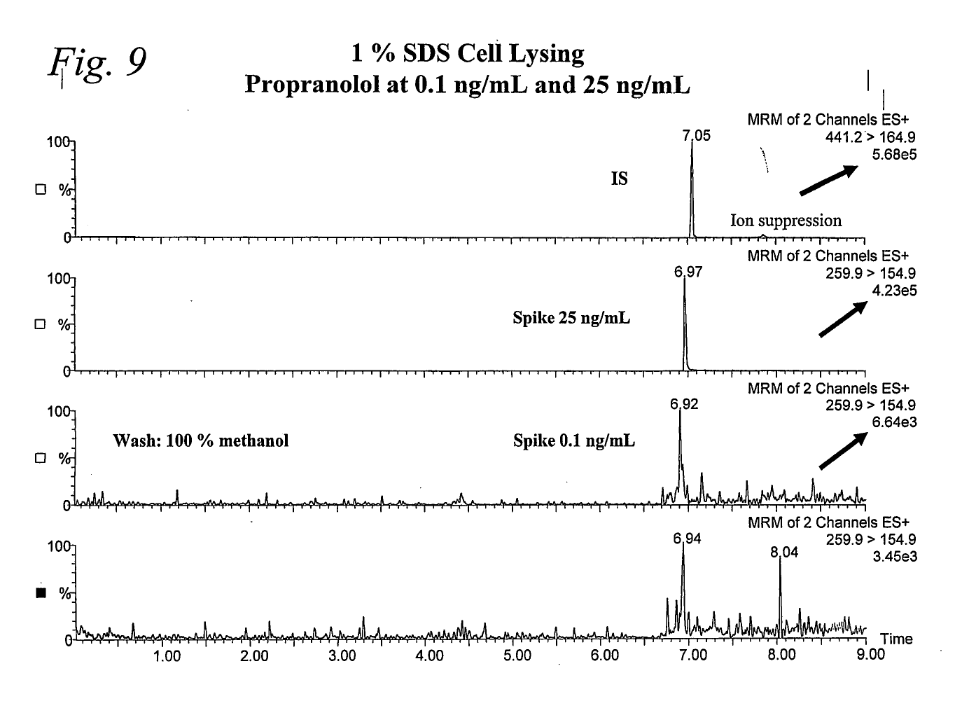

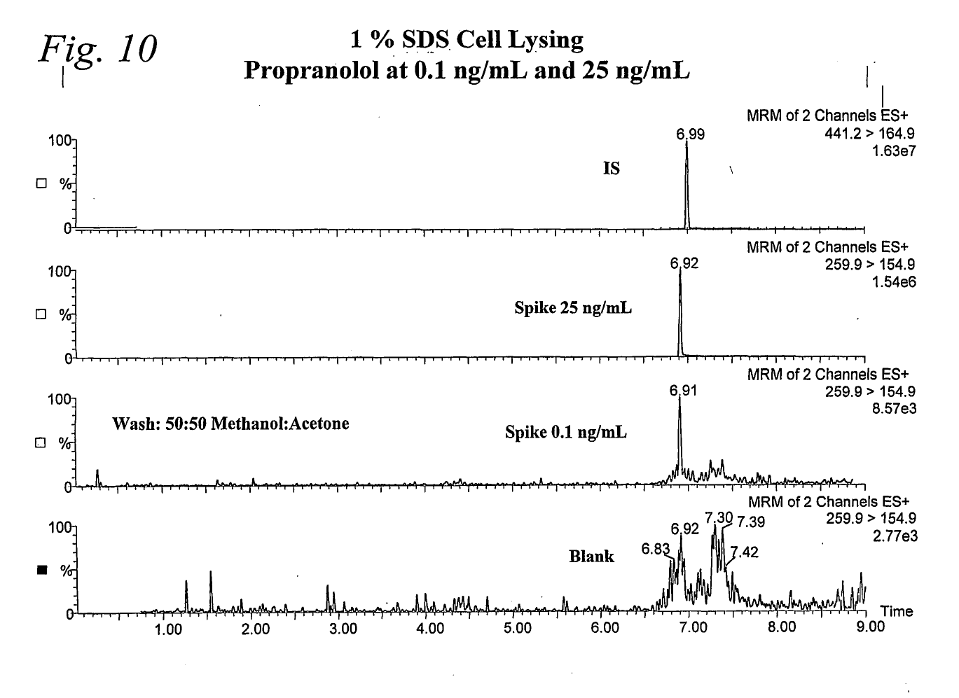

Washing step # 2: The effluent of pump B was then directed toward the Oasis MCX cartridge for additional clean up for one minute and at 4 mL/min. As the propranolol was now locked onto the ion exchanger, a “stronger” solvent was used to remove various types of possible suppressants. For example, cell samples lysed with Triton X-100 required a 100% methanol (polar solvent) wash for an effective clean up protocol, the analysis of which is depicted in FIG. 8. However, cell samples lysed with SDS using 100% methanol as wash # 2 was not effective to remove all traces of SDS, the analysis of which is depicted in FIG. 9. The chromatogram showed a reduced signal intensity, which translates to the presence of trace amounts of SDS during the elution phase (see elution step # 2). When the washing # 2 mobile phase is replaced with a mixture of 50/50 acetonitrile and acetone (less polar solvent), the signal intensity of propranolol are slightly increased, as shown in FIG. 10.

Elution step # 1: The effluent of pump B was then directed toward the Oasis MCX cartridge for elution of propranolol onto the Oasis HLB cartridge. The mobile phase of pump B was 100% methanol plus 2% ammonium hydroxide and set at 4 mL/min. This mobile phase was used to neutralize the drug from the ion exchanger of MCX and the high organic level was use for total elution from the sorbent. At this point, if the effluent was to be directed to a C18 column, the drug would have no retention because of high level of organic (partition coefficient would favor the mobile phase rather than the stationary phase). To overcome this problem, the effluent coming out of the MCX has to be changed to a low organic content. By adding a Tee and an Oasis HLB cartridge, the high organic effluent from MCX was diluted by a ratio of 2:1 with water and the drug was retrapped on the Oasis sorbent as a neutral species. This step was done for one minute.

Elution step # 2: As a final step, the gradient pump of 2690 eluted the propranolol trapped on the Oasis HLB onto an XTerra C18 column for further peak focusing.

Replacing the surfactants above with ALS, this experiment was repeated. The results are discussed below.

### CONCLUSIONS

Although the presence of propranolol was detectable through UV analysis in the solid phase extraction, the MS analysis showed only low levels of the propranolol, if any, due to ion suppression by SDS or Triton X100. FIG. 1 shows a comparison of the MS analysis of the cell lysates using ALS as compared with SDS.

Incorporation by Reference

The entire contents of all patents, published patent applications and other references cited herein are hereby expressly incorporated herein in their entireties by reference.

Equivalents

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A method for analysis of a small molecule comprising contacting a sample containing at least one small molecule with a surfactant represented by the formula: 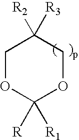
in which
p is 0, 1 or 2;
R is alkyl;
R₁ and R₂ are each, independently, hydrogen or methyl; and
R₃ is selected from —OSO₃⁻, —R₄OSO₃⁻, —R₄OR₅SO₃⁻, and —OR₅SO₃⁻,
wherein R₄ and R₅ are each, independently, lower alkyl; to thereby analyze the small molecule.

2. The method of claim 1, wherein the sample is a biological sample.

3. The method of claim 2, wherein the biological sample comprises one or more cells.

4. The method of claim 3, wherein the biological sample comprises a tissue culture.

5. The method of claim 3, wherein the biological sample comprises a biological fluid, a biological tissue, a biological matrix, an embedded tissue sample, a cell culture supernatant, or combination thereof.

6. The method of claim 2, wherein the analysis comprises lysis of the cell.

7. The method of claim 2, wherein the analysis comprises clarification

8. The method of claim 2, wherein the analysis comprises clarification of tissue culture supernatant.

9. The method of claim 2, wherein the analysis comprises dissociation of a small molecule from a biological matrix.

10. The method of claim 2, wherein the biological fluid is selected from the group consisting of blood, blood plasma, urine, spinal fluid, mucosal tissue secretions, tears, interstitial fluid, synovial fluid, semen, and breast milk.

11. The method of claim 1, wherein the analysis comprises isolation of the small molecule.

12. The method of claim 1, wherein the analysis is selected from the group consisting of solid phase extraction, solid phase micro extraction, electrophoresis, mass spectrometry, liquid chromatography, liquid-liquid extraction, membrane extraction, soxhlet extraction, precipitation, clarification, electrochemical detection, staining, elemental analysis, Edmund degradation, nuclear magnetic resonance, infrared analysis, flow injection analysis, capillary electrochromatography, ultraviolet detection, and combinations thereof.

13. The method of claim 1, wherein the small molecule is selected from the group consisting of a drug, a prodrug, a metabolite of a drug, and a product of a reaction associated with a natural biological process.

14. The method of claim 1 wherein the analysis comprises high performance liquid chromatography.

15. The method of claim 1 wherein the analysis comprises solid phase extraction.

16. The method of claim 1 wherein the analysis comprises mass spectrometric detection.

17. A method for performing cell lysis comprising contacting a cell containing at least one small molecule with a surfactant represented by the formula (Formula I): 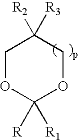
in which
p is 0, 1 or 2;
R is alkyl;
R₁ and R₂ are each, independently, hydrogen or methyl; and
R₃ is selected from —OSO₃⁻, —R₄OSO₃⁻, —R₄OR₅SO₃⁻, and —OR₅SO₃⁻,
wherein R₄ and R₅ are each, independently, lower alkyl;
to thereby lyse the cell.

18. The method of claim 17 comprising the further step of degrading the surfactant after cell lysis.

19. The method of claim 18 wherein the step of degrading the surfactant after cell lysis comprises contacting the surfactant with an acidic solution.

20. The method of claim 18 comprising the further step of isolating the small molecule.

21. The method of claim 20 comprising the further step of purifying the small molecule.

22. The method of claim 20, wherein the purification step is accomplished by solid phase extraction or HPLC.

23. The method of claim 17 wherein the surfactant is represented by the following formula: 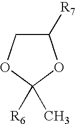
in which
R₆ is alkyl;
R₇ is selected from —OSO₃⁻, —R₄OSO₃⁻, —R₄OR₅SO₃⁻, and —OR₅SO₃⁻,
wherein R₄ and R₅ are each, independently, lower alkyl.

24. The method of claim 17 wherein the surfactant has the following chemical structure:

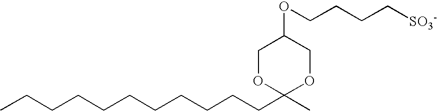

25. The method of claim 17 wherein the surfactant has the following chemical structure:

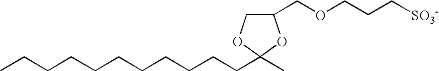

26. A kit for performing cell lysis on a cell containing at least one small molecule comprising:
a surfactant represented by the formula: 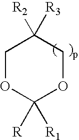
in which
p is 0, 1 or 2;
R is alkyl;
R₁ and R₂ are each, independently, hydrogen or methyl; and
R₃ is selected from —OSO₃⁻, —R₄OSO₃⁻, —R₄OR₅SO₃⁻, and —OR₅SO₃⁻,
wherein R₄ and R₅ are each, independently, lower alkyl; and instructions for use.

27. The kit of claim 26 further comprising a solution for degrading the surfactant.

28. The kit of claim 26 further comprising a solid phase extraction device.

29. The kit of claim 26 wherein the surfactant is represented by the following formula: 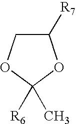
in which
R₆ is alkyl;
R₇ is selected from —OSO₃⁻, —R₄OSO₃⁻, —R₄OR₅SO₃⁻, and —OR₅SO₃⁻,
wherein R₄ and R₅ are each, independently, lower alkyl.

30. The kit of claim 26 wherein the surfactant has the following chemical structure:

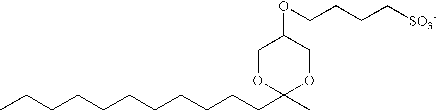

31. The kit of claim 26 wherein the surfactant has the following chemical structure:

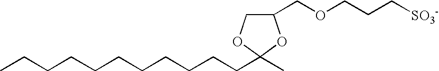

32. A method for eletrophoretically isolating a small molecule from a sample comprising contacting a sample containing at least one small molecule with a surfactant represented by the formula (Formula I): 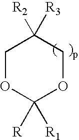
in which
p is 0, 1 or 2;
R is alkyl;
R₁ and R₂ are each, independently, hydrogen or methyl; and
R₃ is selected from —OSO₃⁻, —R₄OSO₃⁻, —R₄OR₅SO₃⁻, and —OR₅SO₃,
wherein R₄ and R₅ are each, independently, lower alkyl;
to form a sample/surfactant complex,
performing electrophoresis on the sample/surfactant complex,
to thereby electrophoretically isolate the small molecule.

33. The method of claim 32 comprising the further step of degrading the surfactant after electrophoresis.

34. The method of claim 33 wherein the step of degrading the surfactant after electrophoresis comprises contacting the surfactant with an acidic solution.

35. The method of claim 33 comprising the further step of purifying the small molecule.

36. The method of claim 32 wherein the surfactant is represented by the following formula: 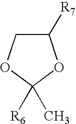
in which
R₆ is alkyl;
R₇ is selected from —OSO₃⁻, —R₄OSO₃⁻, —R₄OR₅SO₃⁻, and —OR₅SO₃⁻,
wherein R₄ and R₅ are each, independently, lower alkyl.

37. The method of claim 32 wherein the surfactant has the following chemical structure:

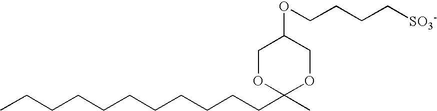

38. The method of claim 32 wherein the surfactant has the following chemical structure:

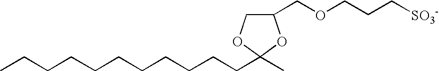

39. A kit for performing electrophoresis on a sample containing at least one small molecule comprising:
a surfactant represented by the formula: 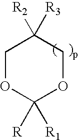
in which
p is 0, 1 or 2;
R is alkyl;
R₁ and R₂ are each, independently, hydrogen or methyl; and
R₃ is selected from —OSO₃⁻, —R₄OSO₃⁻, —R₄OR₅SO₃⁻, and —OR₅SO₃⁻,
wherein R₄ and R₅ are each, independently, lower alkyl; and instructions for use.

40. The kit of claim 39 further comprising a solution for degrading the surfactant.

41. The kit of claim 39 further comprising a molecular weight standard.

42. The kit of claim 39 further comprising a staining reagent.

43. The kit of claim 39 wherein the surfactant is represented by the following formula: 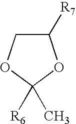
in which
R₆ is alkyl;
R₇ is selected from —OSO₃⁻, —R₄OSO₃⁻, —R₄OR₅SO₃⁻, and —OR₅SO₃⁻,
wherein R₄ and R₅ are each, independently, lower alkyl.

44. The kit of claim 39 wherein the surfactant has the following chemical structure:

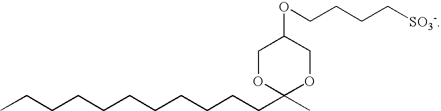

45. The kit of claim 39 wherein the surfactant has the following chemical structure:

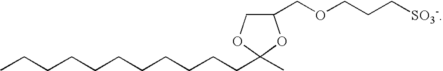

46. A method of solubilizing a small molecule comprising contacting a sample containing at lease one small molecule with a surfactant represented by the formula (Formula I): 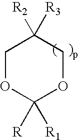
in which
p is 0, 1 or 2;
R is alkyl;
R₁ and R₂ are each, independently, hydrogen or methyl; and
R₃ is selected from —OSO₃⁻, —R₄OSO₃⁻, —R₄OR₅SO₃⁻, and —OR₅SO₃⁻,
wherein R₄ and R₅ are each, independently, lower alkyl; to thereby solubilize the molecule.

47. A method of regenerating a liquid chromatography column having a sorbent to which is bound at least one small molecule comprising contacting the sorbent with a surfactant represented by the formula (Formula I): 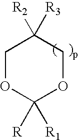
in which
p is 0, 1 or 2;
R is alkyl;
R₁ and R₂ are each, independently, hydrogen or methyl; and
R₃ is selected from —OSO₃⁻, —R₄OSO₃⁻, —R₄OR₅SO₃⁻, and —OR₅SO₃⁻,
wherein R₄ and R₅ are each, independently, lower alkyl
such that the small molecule bound to the sorbent is removed, thereby regenerating the column.

48. A method for analyzing a small molecule contained in a cell comprising:
contacting the cell with a surfactant represented by the formula (Formula I): 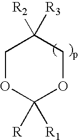
in which
p is 0, 1 or 2;
R is alkyl;
R₁ and R₂ are each, independently, hydrogen or methyl; and
R₃ is selected from —OSO₃⁻, —R₄OSO₃⁻, —R₄OR₅SO₃⁻, and —OR₅SO₃⁻,
wherein R₄ and R₅ are each, independently, lower alkyl; to lyse the cell; and
analyzing the small molecule.

49. The method of claim 48, wherein the step of analyzing comprises mass spectrometry.

50. The method of claim 48, wherein the step of analyzing comprises electrophoresis.

51. The method of claim 48, wherein the small molecule is propranolol.
